(19) United States
(12) Patent Application Publication
(10) Pub. No.: US 2003/0229326 A1
(43) Pub. Date: Dec. 11, 2003

(54) Hydrophilic meltblown pad

(76) Inventors: Edward Hovis, Newark, DE (US); Roger Davis, Elkton, MD (US); James Dickson, Yardley, PA (US); Ashish Mathur, Newark, DE (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 10/163,397

(22) Filed: Jun. 5, 2002

(51) Int. Cl. A61F 13/15; A61F 13/20

(52) U.S. Cl. 604/370

(57) ABSTRACT

An absorbent article includes a fluid-permeable cover sheet; a fluid-permeable or liquid impermeable back sheet; and a nonwoven fabric between the cover sheet and the back sheet, the nonwoven fabric including an absorbent assembly of meltblown thermoplastic fibers and a hydrophilic wetting agent, wherein the cover sheet, nonwoven fabric and back sheet are bonded together. The article is particularly suitable for use as a bandage or wound dressing.

### BACKGROUND OF THE INVENTION

1. Field of Invention

The invention relates generally to absorbent laminates and to methods of making same, and more particularly to bandages including such absorbent laminates.

2. Description of Related Art

Absorbent non-stick pads employed as the absorbent components in high quality adhesive bandages preferably include an apertured web laminated to a nonwoven absorbent material. In one conventional structure, the apertured web is a high density polyethylene web sold under the trademark DELNET by DelStar Technologies Inc. of Middletown, Del., (formerly Applied Extrusion Technologies), and the nonwoven absorbent material is a needle-punched web including a blend of polypropylene and rayon fibers. In this structure, the DELNET web has anti-stick properties relative to body wounds to be covered, and the nonwoven absorbent material has a weight of approximately 3.7 ounces/yard² (125 gm/m²). These two components are heat-sealed together in a bonding nip under pressure to ensure that the components are effectively bonded together.

While the above-described structure has functioned in a satisfactory manner in bandage applications, that structure is undesirably expensive to fabricate, particularly because of the relatively high cost of the needle-punched nonwoven absorbent component of the pad. Also, there are major concerns over the cleanliness of the needle-punched nonwovens, because of the several manufacturing processes involved. In the first stage, staple fibers are typically produced using extrusion-based processes such as dry spinning, wet spinning or melt spinning. Both dry spinning and wet spinning involve the use of harsh solvents, whereas melt spinning is relatively clean, as the fiber is directly extruded from virgin polymer. In the second stage, these fibers typically go through a carding process to open and parallelize the fibers. The carded web is finally converted into the nonwoven form by using mechanical entanglement or by thermal means. Synthetic fibers are essentially hydrophobic in nature and are therefore typically coated with a lubricant or spin finish for ease in processing. A common problem with needle-punched nonwoven is contamination by lubricating oil or grease from the needle loom and the presence of broken needles. Both contaminants are inherent in the needling process. The stringent FDA and toxicity regulations for materials used in wound care applications therefore impose severe restrictions on the manufacturing practices.

It is known that for a given weight of fibers a pad having greater loft or thickness will have the greatest absorbency. It also is known that increasing the weight of fibers present in the pad can increase absorbency. However, this latter-mentioned approach for increasing absorbency increases the cost of fabricating the pad by increasing the cost of materials utilized in the pad.

Another limitation of the above process is that, the pressure required at the bonding nip to effect bonding of the nonwoven absorbent component to the apertured web, while controlling, or preventing, shrinkage of the oriented web, results in the formation of an absorbent pad that has a high fiber density and little entrapped air. This undesirably limits the absorbent capacity of the pad besides making the process somewhat difficult to control.

It is known to use multi-layer constructions or bicomponent fibers to obviate the problem of shrinkage during the bonding of the various layers to each other. The prior art also includes numerous additional disclosures of multi-layer and/or multi-fiber fabrics wherein one or more of the fibers/layers is a thermoplastic material that functions as a bonding agent in the fabric. See, e.g., U.S. Pat. No. 5,362,546 to Boulanger, U.S. Pat. No. 4,545,372 to Lauritzen, U.S. Pat. No. 4,041,203 to Brock et al., U.S. Pat. No. 3,846,205 to Yazawal, U.S. Pat. No. 4,657,802 to Morman, U.S. Pat. No. 4,726,976 to Karami et al., U.S. Pat. No. 4,214,582 to Patel, U.S. Pat. No. 3,285,245 to Eldredge et al. and U.S. Pat. No. 5,114,787 to Chapline et al.

U.S. Pat. No. 4,348,445 to Craig discloses a laminate that includes an unoriented film formed of a propylene/1-butene copolymer bonded to a netting layer including oriented fibers. This patent discloses laminates wherein two of such film layers are bonded to a central netting layer, and wherein two netting layers are bonded to a central film layer. The selection of materials allegedly prevents shrinkage or distortion of the oriented netting layer(s) during the bonding of the multiple layers into a laminate construction. However, the use of multilayers aggravate the cleanliness problem even further as well as increase the overall cost of the product.

It is also known to prepare laminated constructions including a fluid-pervious, oriented plastic substrate bonded to a meltblown nonwoven absorbent web. For example, U.S. Pat. No. 5,643,240 to Jackson et al. discloses a combination apertured film and lofty fibrous nonwoven web separation layer which is said to be particularly well suited for use as a body side liner for personal care absorbent articles such as sanitary napkins, bandages and the like. In addition, U.S. Pat. No. 4,957,795 to Riedel discloses an absorbent elastomeric wound dressing comprising (1) a fluid permeable, compliant, low adherency wound contacting layer, (2) an intermediate conformable, fluid-absorbent element, the element having a nonwoven fibrous matrix of elastomeric meltblown small diameter fibers and absorbent staple fibers or absorbent particulate material, wicking staple fibers, and bulking staple fibers dispersed throughout the matrix, and (3) a soft, compliant cover layer.

Finally, U.S. Pat. No. 5,130,073 to Meirowitz et al. and U.S. Pat. No. 5,439,734 to Everhart et al. disclose treating hydrophobic fibers to increase their hydrophilicity, thus rendering the fibers more suitable for use in products designed to absorb aqueous fluids.

Despite the foregoing developments, it is desired to provide alternative laminates having unique structural and functional characteristics.

All references cited herein are incorporated herein by reference in their entireties.

### BRIEF SUMMARY OF THE INVENTION

Accordingly, the invention provides an absorbent article comprising:

a fluid-permeable cover sheet;

a back sheet which is fluid permeable or liquid impermeable; and

a nonwoven fabric between the cover sheet and the back sheet, the nonwoven fabric comprising an absorbent assembly of meltblown thermoplastic fibers and a hydrophilic wetting agent,

wherein the cover sheet, the nonwoven fabric and the back sheet are bonded together.

Also provided is a process for providing an absorbent article, said process comprising:

providing a fluid-permeable cover sheet;

providing a back sheet which is fluid-permeable or liquid impermeable;

providing a nonwoven fabric between the cover sheet and the back sheet, the nonwoven fabric comprising an absorbent assembly of meltblown thermoplastic fibers and a hydrophilic wetting agent; and

bonding together the cover sheet, the nonwoven fabric and the back sheet to produce the absorbent article.

### BRIEF DESCRIPTION OF SEVERAL VIEWS OF THE DRAWINGS

The invention will be described in conjunction with the following drawings in which like reference numerals designate like elements and wherein:

FIG. 1A shows an exploded top view of an embodiment of the absorbent article of the invention; and

FIG. 1B shows a side view of the embodiment of FIG. 1A as assembled.

### DETAILED DESCRIPTION OF THE INVENTION

In preferred embodiments, the invention provides an absorbent laminate comprising a fluid-permeable (preferably liquid-permeable), plastic cover sheet (preferably apertured) bonded to a nonwoven absorbent substrate producing using a meltblowing process. The meltblown process is the cleanest and most direct method of producing a nonwoven fabric from virgin polymer.

Meltblown webs are produced using an apparatus similar to the apparatus discussed, for example, in Wente, Van A., “Superfine Thermoplastic Fibers,” Industrial Engineering Chemistry, Vol. 48, pp. 1342-1346 and in Wente, Van A., “Manufacture of superfine organic fibers,” Report No. 4364 of the Naval Research Laboratories, May 25, 1954. The meltblowing process essentially involves extruding a thermoplastic fiber-forming polymer, preferably polypropylene, with extremely low viscosities (high melt flow rates, in the range of 800 to 1500 or higher) through a linear die containing hundreds of fine orifices. Convergent streams of hot air contact the polymer at the die tip and rapidly attenuate the extruded polymer streams to form extremely fine fibers. The fibers entangle and stick to each other as they cool down and are deposited on a collector screen or forming belt as a fine-fiber, self-bonded meltblown web. Fiber diameters in a meltblown web range between 0.5 and 25 microns, but are typically in the range of 1 to 5 microns. The small fiber diameter and high uniformity of meltblown webs provide very desirable attributes for filter media such as high opacity and surface area, high filtration efficiency, good barrier properties and wicking ability. Unique properties can be engineered into a meltblown web by controlling key physical attributes such as basis weight, thickness, air permeability and fiber size and distribution.

Suitable fibers for forming the nonwoven will typically include thermoplastic fibers such as those made from polyolefins, polyesters and nylon. Meltblown webs made from polyester polymers are preferred when gamma sterilization is required for the end product. Polyolefin nonwoven fabrics, such as carded webs, spunbond, meltblown or composites thereof, are preferred as components in wound care pads, adhesive bandages (e.g., BAND-AIDS), sanitary articles, such as single use diapers, feminine hygiene products and incontinence care products. The recognized benefits of polyolefin based, especially polypropylene, fabrics include the relatively low raw material cost, ease of manufacturing, desirable strength to basis weight ratio and softness.

The meltblown media are preferably rendered hydrophilic by incorporating a judicious combination of hydrophilic wetting agents during the melt blowing process. The permanence of hydrophilicity can be controlled through composition and concentration of the wetting agent.

Wettability or hydrophilicity is generally achieved by incorporating wetting agents either in or on the polymer, fiber or web. Most of the prior art involves chemical modification of the surface of a web using topical treatments such as radiation, grafting and surfactant coating. It is known in the industry that certain surfactants, such as Triton X-100 from Rohm and Haas, can be applied as an aqueous solution or suspension to the surface of hydrophobic fibers, filaments or nonwoven fabrics with the resulting effect of rendering the fibers, filaments or fabrics wettable, although not absorbent. These topical treatments can be applied by any means familiar to one skilled in the art, such as foaming, spraying, dip-and-squeeze or gravure roll. In almost every case, some sort of heating step is required to remove residual water or solvents used to prepare the surfactant solution or suspension. This step adds significantly to the manufacturing costs and complexity. Further, thermoplastics are altered by exposure to heat and careful monitoring of the heating process is required to ensure that fabric properties are not adversely affected.

Nonwoven fabrics made from internally treated polypropylene exhibit properties, which are greatly superior to other internal wetting agents or topical treatments known in the prior art. However, the chemistry and concentration of these wetting agents is extremely important, as most of the commercially available wetting agents are highly migratory and can be leached out. Further, hydrophilic wetting agents can be introduced either in a liquid form, in a pellet or powder form. The most desirable way, as is used in the present invention, is by blending in a desired amount of the wetting agent(s), available in powder or pellet form, at the polymer feed stage. This approach ensures uniform blending and mixing of the wetting agent(s) with the base polymer, prior to being fed into the extruder. Alternatively, the wetting agent(s) can also be introduced into the polymer melt in the form of a liquid. For example, the wetting agent is introduced directly into the extruder, or it may be introduced at a stage between the extruder and the die, where it is mixed and blended with the base polymer in the desired proportion. However, this approach would involve some modification of the existing equipment.

There is an unexpected aesthetic benefit as the fabrics produced from the fibers or filaments of this invention are considerably softer than untreated fabrics. This feature can be measured as a significant change in the coefficient of static or dynamic friction.

The hydrophilic chemical wetting agents incorporated into the filaments used to form the hydrophilic meltblown fabric described above, may include any hydrophilic wetting agent, which is thermally stable at temperatures up to 300° C. and sufficiently phase separates such that the wetting agent migrates from the bulk of the polymer fiber towards the surface of the polymer fiber as the fiber cools without requiring the addition of heat. Once at the polymer surface, the chemical wetting agent changes the hydrophobicity of the polymer surface such that the surface of polymer rapidly wets upon contact with an aqueous fluid. For the embodiments where the hydrophilic properties of the absorbent fabric are due to hydrophilic chemical added on the surfaces of the fabric filaments, the hydrophilic wetting agents may include any wetting agent, which is thermally stable at temperatures up to 100° C. Once on the filament surface, the chemical wetting agent changes the inherent hydrophobicity of the filament surface such that the filament surface wets upon contact with an aqueous fluid. Such chemical wetting agents include, but are not limited to, those identified above and having thermal stabilities up to 100° C. Any such chemical is suitable for the present invention as long as the chemical does not negatively impact desired properties of the product of the invention.

Alternatively, the hydrophilic chemical wetting agent may be coated onto the meltspun fabric. Suitable methods of applying the hydrophilic chemical wetting agents to a meltspun fabric include, but are not limited to, particle coating, spray coating, or solution coating. Such chemical wetting agents include, but are not limited to, one or a combination of wetting agents selected from the following classes of wetting agents: (i) polyoxyalkylene modified fluorinated alkyls, (ii) polyoxyalkylene fatty acid esters and glycerides (iii) polyoxyalkylene modified polydimethyl siloxanes and PEG-terephthalate (polyethylene glycol modified terephthalate) and (iv) ethoxylated alkyl phenols. The choice of one or more chemical wetting agents depends upon, for example, cost, durability, compatibility with the polymeric material, and the overall contribution to the properties of the finished drape. The most durable compounds amongst these are those, which migrate very slowly to the surface of the web after manufacture. The choice of wetting agent that can be used for wound pads and dressings is also limited by the stringent FDA and toxicity regulations of the media. The efficacy of the wetting agents depends on two main factors: a) incorporation of the wetting agent in the melt state, and b) migration of the wetting agents to the surface. These depend upon several factors such as the polymer type, chemistry, solubility, melting point and molecular weight and the diffusion characteristics of the polymer wetting agent.

Wetting agents suitable for use in certain embodiments of the invention are disclosed in WO/0242530A1.

The present invention uses relative high concentrations of admixtures of durable hydrophilic wetting agents such as those produced by Techmer PM of Knoxville, Tenn. under the product names of PPM 11866 and S-219460. Similar performing wetting agents may also be obtained from Ciba Specialty Chemicals (CGX461), Polyvel (VF350), Dow Corning, DuPont, 3M and other wetting agent manufacturers. Alternatively, if the wetting agent is to be used in liquid form, the surfactant can be obtained from various chemical manufactures such as ICI, DuPont, Shell, Dow Corning and others. In general, the formulations include an active chemical provided in a carrier resin, preferably polypropylene, of a given meltflow rate (MFR) suitable for meltblowing. The active ingredient (surfactant) in each of these wetting agents is up to 30% by weight.

In one embodiment of this invention, the meltblown web consists of 90% to 98% polypropylene meltblown resin and 2% to 10% of CGX461X from Ciba Specialty Chemicals. In another embodiment of he invention, the meltblown web consists of 60% to 90% polypropylene meltblown resin, 10% to 30% of PPM 11866, i.e., 3-9% of the active chemical and/or 0% to 10% of the S-219460 wetting agent, i.e., upto 3% of the active chemical. Most preferably, the meltblown web should consist of 75% to 85% of polypropylene meltblown resin, 15% to 20% of PPM 11866, i.e., 4-6% of the active chemical and 3-5% of the S-219460 wetting agent, i.e., 0.9-1.5% of the active chemical.

The degree of hydrophilicity of a media can be determined by measuring the wet-out time; i.e., the time it takes to wet-out the media. The absorbency is measured by measuring the amount of water absorbed over a fixed period of time.

This invention provides a two or three-component laminated construction in the form of a sterile bandage. One or both outer surfaces of the bandage are non-stick surfaces, wherein, the non-stick side of the bandage can be placed adjacent an injured skin area of a person. In accordance with this invention, the absorbent pad or member is a hydrophilic meltblown manufactured having a thickness of approximately 50 mils and having an absorbency of approximately 30 oz/yd² (1010 g/m²) absorbency.

Meltblown webs of the present invention have preferred basis weights ranging from 50 grams/m² (gsm) to 500 gsm, and more preferably in the range of 100 gsm to 400 gsm. Preferred fiber size in the meltblown webs is in the range of 0.5 microns to 20 microns, and more preferably in the range of 0.5 microns to 5 microns. The mean pore diameter of a meltblown web is preferably in the range of 5 microns to 50 microns, and more preferably in the range of 10 microns to 25 microns.

In a preferred embodiment, the absorbent article of the invention comprises a permeable cover layer, which allows fluids to penetrate but has a non-stick surface. Most preferably, the cover layer is an oriented polyethylene web sold under the trademark DELNET by DelStar Technologies, Inc. Such apertured webs, including an identification of various polymers usable in the webs and an identification of methods employed to form such webs, are disclosed in, e.g., U.S. Pat. Nos. 3,137,746, 3,441,638, 4,842,794 and 5,207,962, assigned to Applied Extrusion Technologies, Inc.

A very desirable feature of the present invention is that a pre-consolidated absorbent material, such as the prior art needle-punched web, need not be employed. Rather, the laminated web can be made in a single, continuous in-line process, wherein a hydrophilic meltblown is blown directly onto an apertured film, such as DELNET, to form a two-layered laminate. An additional layer of DELNET or other material may optionally be introduced to form the backing layer of a three-component laminate. The layers of the laminate can be easily bonded together without adversely affecting the high loft absorbent properties of the product by selectively fusing the edges using ultrasonics, rotary die-cutting or hot die-stamping operation. Alternatively, where a higher Z-axis strength is desired, the entire body of the laminate can be bonded ultrasonically using a variety of bond geometries. This arrangement in which fiber escape from the interior of the laminate is prevented is extremely important for certain applications, and in particular for wound dressings.

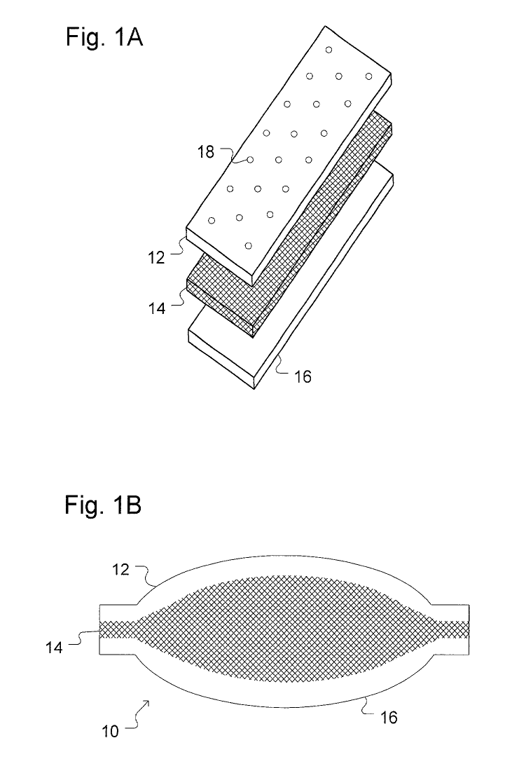

Referring to FIGS. 1A and 1B, a further preferred absorbent article 10 of this invention comprises a multi-component lamination of cover sheet 12 over nonwoven fabric 14, which is over back sheet 16. Cover sheet 12 is fluid permeable and can include pores 18, as shown in the figures. Nonwoven 14 is an interior absorbent member of hydrophilic meltblown, which can optionally include medicaments or other wound treating ingredients therein when article 10 is in the form of a dressing for wounds. Although back sheet 16 is depicted without pores, it is possible in other embodiments for back sheet to include pores. FIG. 1B shows an embodiment wherein the rectangular three-layered laminate of FIG. 1A is bonded along the longest two of its four edges.

The multicomponent absorbent pad is preferably produced in a single continuous process, wherein meltblown fibers are blown directly in the form of a web onto a layer of fluid permeable plastic netting, such as DELNET, followed by introduction of another layer of netting onto the meltblown. The edges of the three-layered composite are then sealed using ultrasonic sealing and the final product wound up in the form of rolls or spools.

In one embodiment of the invention, the plastic substrate is a multi-layer, laminated plastic film or net (preferably formed by coextrusion) wherein the layer of the coextruded substrate contiguous to the nonwoven absorbent member is a low melt temperature, heat-softenable skin that is heat-softenable at a temperature lower than the heat-softening temperature of the nonwoven absorbent member, said low melting skin of the multi-layer oriented plastic substrate providing a bonded connection between the substrate and the nonwoven absorbent member.

In another embodiment of the invention, the backing layer comprises a liquid impermeable film with a high moisture vapor transmission rate (MVTR), preferably greater than 1000 grams/square meters/day measured in accordance with ASTM Test Method E-96D. Nonlimiting examples of such films include Exxon EXXAIR and DELNET Elastic Film (brown or clear).

The invention will be illustrated in more detail with reference to the following Examples, but it should be understood that the present invention is not deemed to be limited thereto.

### EXAMPLES

Unless otherwise specified, the test procedures for evaluating the degree of hydrophilicity were as follows:

Wink Test: Place the burette or medicine dropper (filled with water) 1.2 cm (½ inch) over the full width sample being tested. Drop one drop of water on the sample. Measure the time from the drop of water touching the surface to the time the drop of water is absorbed, leaving a dull wet spot.

Absorbency Test: Cut samples from left, center, and right of product being tested using a 7.62 cm×10.16 cm template, the 10.16 cm length being machine direction (MD). Weigh the test specimens and record (WS). Determine the tare weight of the plastic culture dish (WG) and test basket (WB). Place a specimen into the basket with the 7.62 cm edge parallel to the side of the basket. Hold the basket with the specimen closest to the water and drop the basket on its side from a height of 2.54 cm into the container of water. Allow the basket to remain submerged in the water for ten seconds. Remove the basket and sample from water and allow the water to drain from it for one minute. Weigh the basket and its contents (Wt) in the plastic culture dish. Repeat the above procedure for the remaining test specimens. Record and calculate the capacity of water held by the test sample.

C=Capacity in g/m²=[Wt−(WB+WS+WG)]×129.2

### Example 1

A 100 gsm polypropylene meltblown media was produced on a 12 inch (30.5 cm) wide meltblown line at DELSTAR TECHNOLOGIES, Middletown, Del. using the following formulation:

83% Escorene (Exxon) 3746 polypropylene resin having 1500 meltflow rate (MFR);

15% Techmer Wetting Agent S-219461 (PPM 11866); and

2% Techmer Wetting Agent S-219460.

The process parameters used to produce the meltblown media were as follows:

| | | |
|---|---|---|
| Die Temperature | 475 ° F. | (246° C.) |
| Air Temperature | 475 ° F. | (246° C.) |
| Quench Temperature | 55° F. | (12.8° C.) |
| Polymer Throughput | 40 lbs/hr | (18 kg/hr) |
| Die-Collector Distance | 14 inches | (36 cm) |
| Belt speed | 26 feet/mm | (7.9 m/mm) |
| Vacuum | 2 inches | (5 cm) |
| Process Air | 800 lbs/hr | (363 kg/hr) |
| Quench Air | 125 lbs/hr | (57 kg/hr) |

The following physical properties of the meltblown media produced were obtained:

| | |
|---|---|
| Basis Weight | 100 gsm |
| Thickness | 45 mils (1.1 mm) |
| Wink | Instantaneous |
| Absorbency | 30 oz/yd² (1010 g/m²) |

### Example 2

A 100 gsm polypropylene meltblown media was produced on the 12 inch (30.5 cm) wide meltblown line at DELSTAR TECHNOLOGIES, Middletown, Del. using the following materials:

95% Escorene (Exxon) 3746 polypropylene resin having 1500 meltflow rate (MFR); and

5% CGX-261 Ciba Hydrophilic Wetting agent from Ciba Specialty Chemicals.

The process parameters used to produce the meltblown media were as follows:

| | | |
|---|---|---|
| Die Temperature | 490° F. | (254° C.) |
| Air Temperature | 490° F. | (254° C.) |
| Quench Temperature | 55° F. | (12.8° C.) |
| Polymer Throughput | 40 lbs/hr | (18 kg/hr) |
| Die-Collector Distance | 22 inches | (56 cm) |
| Belt speed | 20 feet/min | (6 m/min) |
| Vacuum | 2 inches | (5 cm) |
| Process Air | 900 lbs/hr | (408 kg/hr) |
| Quench Air | 125 lbs/hr | (57 kg/hr) |

The following physical properties of the meltblown media produced were obtained:

| | |
|---|---|
| Basis Weight | 100 gsm |
| Thickness | 35 mils (2.2 mm) |
| Wink | Instantaneous |
| Absorbency | 40 oz/yd² (1347 g/m²) |

### Example 3

A 150 gsm three component absorbent laminate of polypropylene meltblown and Delnet X-540E was produced on the 12 inch (30.5 cm) wide meltblown line at DELSTAR TECHNOLOGIES, Middletown, Del. by blowing the meltblown directly onto the Delnet. Another layer of Delnet X 540E was introduced on top of the meltblown and the edges were sealed using ultrasonics. The materials used to prepare the meltblown layer were as follows:

97.5% Escorene (Exxon) 3746 polypropylene resin having 1500 meltflow rate (MFR); and

3.5% CGX-261 Ciba Hydrophilic Wetting agent from Ciba Specialty Chemicals.

The following physical properties of the meltblown media produced were obtained:

| | |
|---|---|
| Basis Weight | 150 gsm |
| Thickness | 90 mils (2.3 mm) |
| Wink | Instantaneous |
| Absorbency | 43 oz/yd² (1448 g/m²) |

While the invention has been described in detail and with reference to specific examples thereof, it will be apparent to one skilled in the art that various changes and modifications can be made therein without departing from the spirit and scope thereof.

What is claimed is:

1. An absorbent article comprising:
a fluid-permeable cover sheet;
a back sheet which is fluid-permeable or liquid impermeable; and
a nonwoven fabric between the cover sheet and the back sheet, the nonwoven fabric comprising an absorbent assembly of meltblown thermoplastic fibers and a hydrophilic wetting agent,
wherein the cover sheet, the nonwoven fabric and the back sheet are bonded together.

2. The article of claim 1, wherein the cover sheet comprises an apertured thermoplastic web.

3. The article of claim 1, wherein the cover sheet comprises an apertured coextruded web.

4. The article of claim 1, wherein the thermoplastic fibers are polypropylene fibers.

5. The article of claim 1, wherein the hydrophilic wetting agent is at least one member selected from the group consisting of polyoxyalkylene modified fluorinated alkyls, polyoxyalkylene fatty acid esters, glycerides, polyoxyalkylene modified polydimethyl siloxanes, polyethylene glycol modified terephthalates and ethoxylated alkyl phenols.

6. The article of claim 1, wherein the hydrophilic wetting agent is distributed throughout a transverse cross-section of each of the fibers.

7. The article of claim 1, wherein the hydrophilic wetting agent is concentrated at a surface of each of the fibers.

8. The article of claim 1, wherein the back sheet comprises an apertured thermoplastic web.

9. The article of claim 1, wherein the back sheet comprises a liquid impermeable film having a moisture vapor transmission rate greater than 1000 grams/square meters/day in accordance with ASTM E96D.

10. The article of claim 1, wherein each of the cover sheet and the back sheet comprises an apertured thermoplastic web.

11. The article of claim 1, wherein the nonwoven fabric consists essentially of the meltblown thermoplastic polyolefin fibers and the hydrophilic wetting agent.

12. The article of claim 1, wherein the article is a bandage and at least one of the cover sheet and the back sheet is adapted to avoid adhering to wounds.

13. The article of claim 1, wherein the cover sheet, the nonwoven fabric and the back sheet are bonded together at points across their entire contacting surfaces.

14. The article of claim 1, wherein the cover sheet, the nonwoven fabric and the back sheet are bonded together only along their edges.

15. The article of claim 10, further comprising adhesive adapted to adhere to skin.

16. A process for producing the absorbent article of claim 1, said process comprising:
providing the fluid-permeable cover sheet;
providing the back sheet which is fluid-permeable or liquid impermeable;
providing the nonwoven fabric between the cover sheet and the back sheet, the nonwoven fabric comprising an absorbent assembly of meltblown thermoplastic fibers and a hydrophilic wetting agent; and
bonding together the cover sheet, the nonwoven fabric and the back sheet to produce the absorbent article.

17. The process of claim 16, wherein the nonwoven fabric is provided between the cover sheet and the back sheet by the following steps:
providing a fiber-forming composition containing a thermoplastic polymer;
meltblowing the fiber-forming composition to deposit fibers directly on the cover sheet and provide the nonwoven fabric on the cover sheet;
providing the back sheet on a surface of the nonwoven fabric opposite the cover sheet.

18. The process of claim 16, wherein the nonwoven fabric is provided as a preformed sheet between the cover sheet and the back sheet.

19. The process of claim 16, wherein each of the cover sheet and the back sheet comprises an apertured thermoplastic web.

20. The process of claim 16, wherein the cover sheet is an apertured fluid permeable sheet and the back sheet is a liquid impermeable sheet.

21. The process of claim 16, wherein the thermoplastic polymer is polypropylene, polyester or nylon.

22. The process of claim 16, wherein the hydrophilic wetting agent is at least one member selected from the group consisting of polyoxyalkylene modified fluorinated alkyls, polyoxyalkylene fatty acid esters, glycerides, polyoxyalkylene modified polydimethyl siloxanes, polyethylene glycol modified terephthalates and ethoxylated alkyl phenols.

23. The process of claim 16, wherein the hydrophilic wetting agent is distributed throughout a transverse cross-section of each of the fibers.

24. The process of claim 16, wherein the hydrophilic wetting agent is concentrated at a surface of each of the fibers.

25. The process of claim 16, wherein the hydrophilic wetting agent is mixed with the thermoplastic polymer in the fiber-forming composition prior to extruding the fibers from an extruder.

26. The process of claim 16, wherein the hydrophilic wetting agent is mixed with the thermoplastic polymer in the fiber-forming composition in an extruder.

27. The process of claim 16, wherein the hydrophilic wetting agent is mixed with the thermoplastic polymer in the fiber-forming composition between an extruder and a die.

28. The process of claim 16, wherein the hydrophilic wetting agent is sprayed onto the nonwoven fabric.

29. The process of claim 16, wherein the bonding is accomplished using ultrasonics, rotary die-cutting or hot die-stamping.

30. The process of claim 16, wherein the bonding is accomplished without any adhesive.

31. The process of claim 16, wherein the cover sheet, the nonwoven fabric and the back sheet are bonded together at points across their entire contacting surfaces.

32. The process of claim 16, wherein the cover sheet, the nonwoven fabric and the back sheet are bonded together only along their edges.

33. The process of claim 16, further comprising applying a skin adhesive to an external surface of the absorbent article.
